(19) United States
(12) Patent Application Publication
(10) Pub. No.: US 2023/0390355 A1
(43) Pub. Date: Dec. 7, 2023

(54) COMPOSITIONS AND METHODS RELATING TO ALOPECIA

(71) Applicant: ALASTIN SKINCARE, INC., Carlsbad, CA (US)

(72) Inventors: Alan David WIDGEROW, Irvine, CA (US); John A. GARRUTO, Encinitas, CA (US)

(73) Assignee: ALASTIN SKINCARE, INC., Carlsbad, CA (US)

(21) Appl. No.: 18/234,795

(22) Filed: Aug. 16, 2023

Related U.S. Application Data
(63) Continuation of application No. PCT/US22/16409, filed on Feb. 15, 2022.
(60) Provisional application No. 63/150,411, filed on Feb. 17, 2021.

(51) Int. Cl. A61K 38/06; A61K 38/08; A61K 38/17; A61Q 7/00; A61K 38/30; A61K 31/519

(52) U.S. Cl. CPC A61K 38/06; A61K 38/08; A61K 38/1783; A61Q 7/00; A61K 38/30; A61K 31/519

(57) ABSTRACT

Provided herein are compositions and methods for targeting dermal white adipose tissue for hair follicle stimulation and regeneration.

### CROSS-REFERENCE

This application is a continuation of International PCT Application No. PCT/US2022/016409 filed Feb. 15, 2022, which application claims benefit of U.S. Provisional Application 63/150,411 filed Feb. 17, 2021, both of which are herein incorporated by reference in their entireties.

### BACKGROUND

Hair loss or alopecia is a common disorder that arises from heterogeneous etiologies. Various treatments are available to prevent or reduce hair loss and stimulate hair growth. However, these treatments may not be the most effective at preventing or reducing hair loss or stimulating hair growth.

### BRIEF SUMMARY

Described herein are methods and compositions for hair follicle stimulation and regeneration. In some instances, methods and compositions as described herein target the dermal white adipose tissue (dWAT). Methods and compositions as described herein targeting the dWAT, in some instances, comprise a peroxisome proliferator-activated receptor (PPAR)-gamma (PPAR-gamma) agonist, an activator of PGC-1α, a metabolic influencer of dWAT lipolysis, a M2 macrophage-polarizing compound, a topical activator of insulin growth factor 1 (IGF-1), an activator or stimulator of autophagy, one or more peptides, or combinations thereof.

An aspect described herein is a composition for targeting dermal white adipose tissue (dWAT) for stimulating or regenerating a hair follicle comprising: (a) a tripeptide-1; (b) a hexepaptide-12; and (c) a PPAR-gamma agonist, wherein the composition stimulates or regenerates the hair follicle. In one feature, the tripeptide-1 comprises palmitoyl tripeptide-1, myristoyl tripeptide-1, or a combination thereof. In one feature, the hexapeptide-12 comprises palmitoyl hexapeptide-12, myristoyl hexapeptide-12, or a combination thereof. In one feature, the composition further comprises hexapeptide-38. In one feature, the hexapeptide-38 is acetyl-hexapeptide-38. In one feature, the hexapeptide-38 is encapsulated in a liposome. In one feature, the composition further comprises an octapeptide. In one feature, the PPAR-gamma agonist is thiazolidinedione (TZD), aleglitazar, farglitazar, muraglitazar, tesaglitazar, or combinations thereof. In one feature, the PPAR-gamma agonist is derived from a plant source. In one feature, the PPAR-gamma agonist is a natural product. In one feature, the composition further comprises a M2-macrophage-polarizing compound. In one feature, the M2-macrophage-polarizing compound is a flavonoid, terpenoid, glycoside, lignan, coumarin, alkaloid, polyphenol, quinone, or combinations thereof. In one feature, the M2-macrophage-polarizing compound is Arctigenin. In one feature, the M2-macrophage-polarizing compound is lupeol, malibatol A, geraniin, aloe-emodin, quercetin, curcumin, apigenin, tacrolimus, or niacinamide, or combinations thereof. In one feature, the M2-macrophage-polarizing compound is derived from a plant source. In some features, the plant source is the Arctium lappa plant, curcumin, luteolin, piperine, resveratrol, silibinum, Baicalin or combinations thereof. In one feature, the composition further comprises a topical activator of insulin growth factor 1 (IGF-1). In one feature, the topical activator of insulin growth factor 1 (IGF-1) is capsaicin, isoflavone, or combinations thereof. In one feature, the topical activator of insulin growth factor 1 (IGF-1) is capsaicin. In one feature, the composition further comprises an activator of PGC-1α. In some embodiments, the PPAR-gamma agonist comprises adiponectin or an adiponectin mimetic. In some embodiments, the adiponectin mimetic comprises AdipoRon, ADP355, ADP399, JT003, 6-C-β-D-glucopyranosyl-(2S,3S)-(+)-5, 7, 30, 40-tetrahydroxydihydroflavonol (GTDF), Osmotin, or combinations thereof. In some embodiments, the compostion further comprises a Jak-STAT inhibitor. In some embodiments, the Jak-STAT inhibitor comprises a ruxolitinib, tofacitinib, or combinations thereof.

An aspect described herein is a method for stimulating or regenerating a hair follicle comprising administering a composition as described herein. In one feature, the method stimulates hair growth.

An aspect described herein is a method for preventing or reducing hair loss comprising administering a composition as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic of a hair follicle.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Methods and Compositions for Targeting Dermal White Adipose Tissue

Described herein are methods and compositions for targeting dermal white adipose tissue (dWAT). Targeting dWAT may result in hair follicle stimulation or regeneration. Methods and compositions as described herein, in some embodiments, comprise a PPAR-gamma agonist, an activator of PGC-la, a metabolic influencer of dWAT lipolysis, a M2 macrophage-polarizing compound, a topical activator of insulin growth factor 1 (IGF-1), an activator or stimulator of autophagy, one or more peptides, or combinations thereof. In some embodiments, the one or more peptides is a tripeptide (e.g., tripeptide-1). In some embodiments, the one or more peptides is a hexapeptide (e.g., hexpapetide-12 and hexapeptide-38). In some embodiments, the one or more peptides is an octapeptide (e.g., GPHGVRQA).

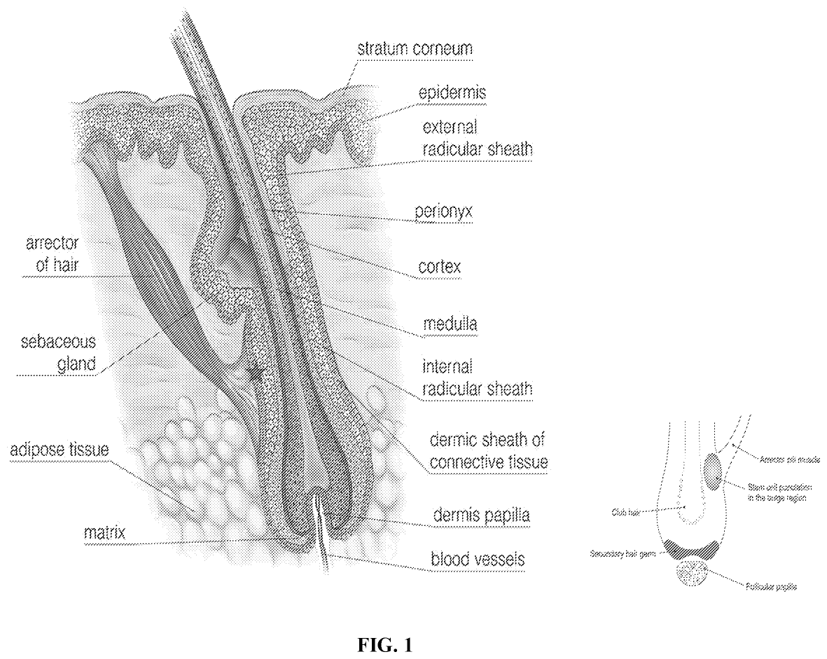

Signaling mechanisms and cellular cross talk occur in the entire microenvironment and macroenvironment surrounding the hair follicle. Particularly, progenitor cells in the bulge area and the dermal papilla may play an important role in regeneration of the hair follicle. Here the signals can coalesce and influence hair follicle stem cells that orchestrate hair regeneration. Bulge stem cells are important for the cyclic regeneration of hair follicles, during which it switches from phases of growth (anagen) via regression (catagen) to relative quiescence (telogen). Further, signals of the tissue macroenvironment arising from dermal fibroblasts, adipocytes (dWAT), preadipocytes, and nerve fibers may contribute to hair follicle cycling. In both the bulge area and the dermal papilla, the interplay of inductive fibroblasts, myofibroblasts, macrophages, keratinocytes, pericytes and ASCs may play interrelated roles in hair cell homeostasis. Because all of these cell phenotypes may be found in the dWAT enveloping the hair follicle, the dWAT may be the primary organ of influence in this sphere and may play a role in hair follicle pathway and stimulation. See FIG. 1.

Described herein are methods for targeting dermal white adipose tissue (dWAT) comprising administering an active agent through a hair follicle, wherein the active agent is delivered to the dWAT through the hair follicle. In some instances, an active agent of low molecular weight is delivered through the hair follicle through the dWAT. In some instances, the active agent has a molecular weight of no more than about 600 Daltons (Da). In some instances, the active agent has a molecular weight of at least or about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, or more than 1000 Daltons (Da). In some instances, the active agent has a molecular weight of at least or about 1000, 1100, 1200, 1300, 1400, 1500, 1600, 1700, 1800, 1900, 2000, 2100, 2200, 2300, 2400, 2500, 2600, 2700, 2800, 2900, 3000, 4000, 5000, 6000, 7000, 8000, 9000, 10000, or more than 10000 Daltons (Da). In some instances, the active agent has a molecular weight in a range of about 50 to about 1000, about 100 to about 900, about 200 to about 800, about 300 to about 700, or about 400 to about 600 Daltons (Da). Alternatively, or in combination, the active agent is a peptide or other active agent encapsulated in a liposome to improve skin penetration through the hair follicle.

In some embodiments, the composition is a topical composition. In some embodiments, the composition is an aqueous formulation. In some embodiments, the composition is an anhydrous formulation.

Modulators of Lactate Dehydrogenase (Ldh) Activity

Described herein are methods and compositions for targeting modulators of lactate dehydrogenase (Ldh) activity. Ususally, human hair follicles use one or more of aerobic glycolysis and glutaminolysis to power their metabolism. During the metabolic process, hair follicle stem cells in the bulge and hair germ can take up glucose and generate lactate with an increased lactate dehydrogenase (Ldh) activity in the bulge. Often, the activation of Ldh activity or the increase in Ldh activity may stimulate the hair cycle. As such, pharmacological inhibition of pyruvate entry into the mitochondria can increase Ldh activity and accelerate the hair cycle.

There exists several pharmacological pathways to inhibit MPC and thereby increase Ldh activity. For example, TZDs may inhibit MPC activity. For example, glitazones, which are members of the thiazolidinedione family, may be potent inhibitors of MPC. In some cases, mitochondrial pyruvate carrier (MPC) may alter the Ldh activity. Usually, the mitochondrial pyruvate carrier (MPC) is a protein which mediates the import of pyruvate across the inner membrane of mitochondria. In some cases, inhibition of pyruvate oxidation via blockade of the MPC complex increases Ldh activity by providing more substrate for the enzyme to convert to lactate. In turn, this activates the hair cycle and stimulates dormant follicles. In some cases, autophagy may be elevated throughout the anagen phase of the hair growth cycle. In some cases, autophagy induction by the longevity metabolites α-KG and α-KB, and small molecule mTOR inhibitors and AMPK activators, may initiate hair follicle activation and hair regeneration.

In some embodiments, the modulator of Ldh activity is a member of the thiazolidinedione (TZD) family. In some embodiments, the modulator of Ldh activity is a TZD. In some embodiments, the modulator of Ldh activity is a glitazone.

PPAR-Gamma Agonists

Described herein are methods and compositions for targeting dWAT to simulate hair follicles and promote hair follicle regeneration. In some embodiments, the methods and compositions comprise an agent that stimulates peroxisome proliferator-activated receptor (PPAR)-gamma (PPAR-gamma). In some embodiments, the agent that stimulates PPAR-gamma is a PPAR-gamma agonist. In some embodiments, the PPAR-gamma agonist is a thiazolidinedione (TZD), aleglitazar, farglitazar, muraglitazar, tesaglitazar, adiponectin, or combinations thereof. Exemplary thiazolidinediones include, but are not limited to, pioglitazone, rosiglitazone, rivoglitazone, and troglitazone. In some embodiments, the PPAR-gamma agonist is derived from a plant source. In some embodiments, the plant source is the tea plant (Camellia sinensis), soybeans (Glycine max), palm oil (Elaeis guineensis), ginger (Zingiber officinale), grapes and wine (Vitis vinifera), or an herb or spice (e.g., Origanum vulgare, Rosmarinus officinalis, Salvia officinalis, Thymus vulgaris). In some embodiments, the PPAR-gamma is a natural product. Exemplary natural products include, but are not limited to, flavonoids (e.g., luteolin, quercetin, kaempferol, catechin, hydroxychalcone, biochanin A, genistein, hydroxydaidzein, and 6′-hydroxy-O-desmethylangolensin), neolignans (e.g., honokiol and magnolol), stilbenes (e.g., resveratrol and amorphastilbol), amorfrutins (e.g., amorfrutin 1, amorfrutin 1, and amorfrutin B), polyacetylenes (e.g., falcarindiol), sesquiterpene lactones (e.g., deoxyelephantopin), and diterpenequinone derivatives (e.g., sargaquinoic acid and sargahydroquinoic acid).

PPAR-Gamma Co Activators

Described herein are methods and compositions for targeting dWAT to simulate hair follicles and promote hair follicle regeneration comprising, in some embodiments, an agent that modulates peroxisome proliferator-activated receptor gamma coactivator 1-alpha (PGC-1α). In some embodiments, the agent that modulates PGC-la is a PGC-la agonist. In some embodiments, the agent that modulates PGC-la is a peptide. In some embodiments, the peptide is hexapeptide-38.

M2 Macrophage—Polarizing Compound

Usually, macrophages are involved in the stimulation of the hair cycle. In some cases, they are involved in matrix scavenging during hair follicle regression, and in hair follicle stem cell activation and anagen induction. In some cases, the macrophage-specific pharmacological inhibition of Wnt production delays hair follicle growth. In some cases, perifollicular macrophages contribute to the activation of skin epithelial stem cells as a novel, additional cue that regulates their regenerative activity. In some cases, M2-phenotype macrophages are involved in stimulating anagen. In some cases, the M2-phenotype macrophase are involved through the IGF-1 pathway.

Described herein are methods and compositions for targeting dWAT to simulate hair follicles and promote hair follicle regeneration comprising, in some embodiments, a M2 macrophage-polarizing compound. In some embodiments, the M2 macrophage-polarizing compound is a flavonoid, terpenoid, glycoside, lignan, coumarin, alkaloid, polyphenol, quinone, or combinations thereof. In some embodiments, the M2 macrophage-polarizing compound is Arctigenin. In some embodiments, the M2 macrophage-polarizing compound is lupeol, malibatol A, geraniin, aloe-emodin, quercetin, curcumin, apigenin, tacrolimus, niacinamide, or combinations thereof. In some embodiments, the M2 macrophage-polarizing compound is derived from a plant source. In some embodiments, the plant is the Arctium lappa plant, curcumin, luteolin, piperine, resveratrol, silibinum, Baicalin or combinations thereof.

Adiponectin

Described herein are methods and compositions for targeting dWAT to stimulate hair follicles and promote hair follicle regeneration comprising adiponectin or an adiponectin mimetic. Sometimes, pharmacological ligands of PPAR-γ may attenuate fibroblast activation and myofibroblast differentiation, ameliorate organ fibrosis while preventing dWAT attrition in vivo. In some embodiments, adiponectin is a PPAR-γ ligand. In some embodiments, adiponectin is a PPAR-γ agonist. In some embodiments, adiponectin stimulates PPAR-γ activity. Usually, adiponectin may be a secreted adipokine. In some cases, adiponectin is about 30 kDa. Targeting cellular adiponectin receptors with synthetic agonist peptides may potentially increase the dWAT compartment with positive effects on hair follicle (HF) growth. In some cases, there are difficulties in targeting adiponectin due to the complex structure of adiponectin, insolubility of adiponectin, and relatively short half-life. In some cases, an adiponectin mimetic that mimics the biological activity of adiponectin may be used. In some embodiments, the adiponectin mimetic is a small molecule. In some embodiments, the adiponectin mimetic is an adiponectin-based short peptide.

In some embodiments, the synthetic agonist peptides include but are not limited to AdipoRon. In some embodiments, AdipoRon has motif similarities with GCPR ligands and AMPK activators. In some embodiments, synthetic agonist peptides include but are not limited to one or more of ADP355, ADP399, and JT003. In some embodiments, plant-based small molecules may function as adiponectin mimetics. In some embodiments, the plant-based small molecules that function as adiponectin mimetics include but are not limited to one or more of 6-C-β-D-glucopyranosyl-(2S,3S)-(+)-5, 7, 30, 40-tetrahydroxydihydroflavonol (GTDF), and Osmotin.

In some embodiments, methods and compositions described herein comprise adiponectin or an adiponectin mimetic. In some embodiments, the methods and compositions provided herein comprise a PPAR-γ ligand. In some embodiments, the PPAR-γ ligand comprises adiponectin or an adiponectin mimetic. In some embodiments, the adiponectin mimetic is a small molecule. In some embodiments, the adiponectin mimetic is an adiponectin receptor agonist. In some embodiments, the adiponectin mimetic is a synthetic small-molecule agonist of the adiponectin receptor 1 (AdipoR1). In some embodiments, the small-molecule agonist is an AdipoRon. In some embodiments, the composition comprises an adiponectin mimetic. In some embodiments, the adiponectin mimetic comprises an adiponectin-based short peptide. In some embodiments, the adiponectin mimetic is ADP355. In some embodiments, the adiponectin mimetic is ADP399. In some embodiments, the adiponectin mimetic is GTDF. In some embodiments, the adiponectin mimetic is JT003. In some embodiments, the adiponectin mimetic is plant-based. In some instances, the plant-based adiponectin mimetic is GTDF. In some instances, the plant-based adiponectin mimetic is Osmotin.

Growth Factors

In some cases, dWAT may regulate human hair growth and pigmentation through the secretion of growth factors. Examples of such growth factors include but are not limited to hepatocyte growth factor (HGF). In some embodiments, growth factors involved in hair growth and pigmentation may be secreted from adipose derived stem cells (ADSCs). In some embodiments, growth factors secreted from ADSC include but are not limited to platelet-derived growth factor (PDGF), vascular endothelial growth factor (VEGF), IGF binding protein precursors, and fibronectin. These growth factors may serve various functions that stimulate hair growth. In some embodiments, PDGF may induce and maintain anagen. In some embodiments, VEGF may increase hair growth by follicle vascularization. In some embodiments, IGF-1 may improve the migration, survival, and proliferation of hair follicle cells.

Described herein are methods and compositions for targeting dWAT to stimulate hair follicles and promote hair follicle regeneration comprising, in some embodiments, activators of growth factors. In some embodiments, the activator of a growth factor is a topical activator of hepatocyte growth factor 1 (HGF-1). In some embodiments, the activator of a growth factor is a topical activator of insulin growth factor 1 (IGF-1). In some embodiments, the topical activator of IGF-1 is capsaicin, isoflavone, vascular endothelial growth factor (VEGF), or TGF-β. In some embodiments, the activator of a growth factor is a topical activator of PDGF. In some embodiments, the topical activator of PDGF is tripeptide-1. In some embodiments, the activator of a growth factor is a topical activator of HGF. In some embodiments, the topical activator of HGF is tripeptide-1. In some embodiments, the activator of a growth factor is octapeptide 45. In some embodiments, the activator of a growth factor is vitamin D.

Peptides

Methods as described herein for targeting dermal white adipose tissue, in some embodiments, comprise administering a composition comprising one or more peptides. In some embodiments, the one or more peptides is encapsulated in a liposome.

In some instances, a peptide is present at about 50 ppm or less to 1000, 5000, 10000, 50000, 100000, 500000 ppm or more, e.g., 100 ppm of the peptide. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % of the first peptide. In some instances, compositions comprise a plurality of peptides. In some instances, a peptide of the plurality of peptides is present at about 50 ppm or less to 1000, 5000, 10000, 50000, 100000, 500000 ppm or more, e.g., 100 ppm of the peptide, or any other suitable amount. The compositions may comprise from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 20 wt. % of the peptide. The amount of peptide in the base can be adjusted up or down.

Compositions as described herein, in some embodiments, comprise a plurality of peptides. In some embodiments, each peptide of the plurality of peptides is provided at least or about 0.00001%, 0.0003%, 0.0005%, 0.001%, 0.001%, 0.005%, 0.0055%, 0.01%, 0.02%, 0.10%, 0.25%, 0.50%, 0.75%, 1.0%, 1.5%, 2.0%, 2.5%, 3.0%, 3.5%, 4.0%, 4.5%, 5.5%, 6.0%, 6.5%, 7.0%, 8%, 9%, 10%, or more than 10% by weight (wt.) In some embodiments, each peptide of the plurality of peptides is provided in a range of about 0.25% to about 10%, about 0.5% to about 8%, about 0.75% to about 6%, or about 1% to about 4% by weight. In some embodiments, each peptide of the plurality of peptides is provided in a range of about 0.001% to about 6%, about 0.002% to about 4%, about 0.01% to about 3%, or about 0.02% to about 2% by weight.

In some embodiments, the peptide is tripeptide-1, hexapeptide-12, an octapeptide, hexapeptide-38, or combinations thereof. In some embodiments, the peptide is functionalized with an acetyl group. For example, the peptide is acetyl hexapeptide-38. In some embodiments, the octapeptide is GPHGVRQA. In some embodiments, the peptide is encapsulated in a liposome.

In some embodiments, the tripeptide-1 is provided at least or about 0.00001%, 0.0005%, 0.001%, 0.001%, 0.005%, 0.0055%, 0.05%, 0.10%, 0.25%, 0.50%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the tripeptide-1 is provided in a range of about 0.25% to about 10%, about 0.5% to about 8%, about 0.75% to about 6%, or about 1% to about 4% by weight.

In some embodiments, the hexapeptide-12 is provided at least or about 0.00001%, 0.0005%, 0.001%, 0.001%, 0.005%, 0.0055%, 0.05%, 0.10%, 0.25%, 0.50%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hexapeptide-12 is provided in a range of about 0.25% to about 10%, about 0.5% to about 8%, about 0.75% to about 6%, or about 1% to about 4% by weight.

In some embodiments, the hexapeptide-38 is provided at least or about 0.00001%, 0.0005%, 0.001%, 0.001%, 0.005%, 0.0055%, 0.01%, 0.02%, 0.05%, 0.10%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hexapeptide-38 is provided in a range of about 0.25% to about 10%, about 0.5% to about 8%, about 0.75% to about 6%, or about 1% to about 4% by weight. In some embodiments, the hexapeptide-38 is provided in a range of about 0.001% to about 6%, about 0.002% to about 4%, about 0.01% to about 3%, or about 0.02% to about 2%. In some embodiments, the hexapeptide-38 is provided in a range of about 0.005% to about 0.02% by weight.

In example embodiments, a weight ratio for the first peptide to the second peptide in a topical formulation is 1 part first peptide to 0.2 to 10 parts second peptide, 1 to 10 parts second peptide, 1 to 8 parts second peptide, or 1 to 5.5 parts second peptide. The following nomenclature is employed herein to refer to various amino acids: Alanine (also referred to herein as “Ala” or “A”), Arginine (also referred to herein as “Arg” or “R”), Asparagine (also referred to herein as “Asn” or “N”), Aspartic acid (also referred to herein as “Asp” or “D”), Cysteine (also referred to herein as “Cys” or “C”), Glutamic acid (also referred to herein as “Glu” or “E”), Glutamine (also referred to herein as “Gln” or “Q”), Glycine (also referred to herein as “Gly” or “G”), Histidine (also referred to herein as “His” or “H”), Isoleucine (also referred to herein as “Ile” or “I”), Leucine (also referred to herein as “Leu” or “L”), Lysine (also referred to herein as “Lys” or “K”), Methionine (also referred to herein as “Met” or “M”), Phenylalanine (also referred to herein as “Phe” or “F”), Proline (also referred to herein as “Pro” or “P”), Serine (also referred to herein as “Ser” or “S”), Threonine (also referred to herein as “Thr” or “T”), Tryptophan (also referred to herein as “Trp” or “W”), Tyrosine (also referred to herein as “Tyr” or “Y”), Valine (also referred to herein as “Val” or “V”).

In some embodiments, the first peptide is a dipeptide. Suitable dipeptides include but are not limited to those having the following sequence of amino acids: KK, KP, CK, KC, KT, DF, NF, VW, YR, or TT. In some embodiments, the dipeptide has the following amino acid sequence: KV. In other embodiments, the first peptide is a tripeptide. Suitable tripeptides include but are not limited to those having the following sequence of amino acids: HGG, RKR, GHK, GKH, GGH, GHG, KFK, or KPK. In some embodiments, the tripeptide has the following amino acid sequence: KVK. In some embodiments, the first peptide is a tetrapeptide. Suitable tetrapeptides include but are not limited to those having the following sequence of amino acids: GQPR, KTFK, AQTR, or RSRK. In some embodiments, the tetrapeptide has the following sequence of amino acids: KDVY. In some embodiments, the second peptide is a pentapeptide. Suitable pentapeptides include but are not limited to those having the following sequence of amino acids: KTTKS, YGGFX, or KLAAK. In some embodiments, the second peptide is a hexapeptide. Suitable hexapeptides include but are not limited to those having the following sequence of amino acids: VGVAPG or GKTTKS. In some embodiments, the hexapeptide has the following sequence of amino acids: FVAPFP. In some embodiments, the second peptide is a heptapeptide. Suitable heptapeptides include but are not limited to one having an amino acid sequence RGYYLLE, or Heptapeptide-6 (a pro-sirtuin peptide). The compositions may include two or more peptides, e.g., two dipeptides and one pentapeptide; one tripeptide and one hexapeptide; one dipeptide, one tripeptide, and one heptapeptide, or the like, provided that the composition contains at least one dipeptide, tripeptide, or tetrapeptide and at least one pentapeptide, hexapeptide, or heptapeptide. In some embodiments, the compositions comprise a tripeptide and one or more hexapeptides. In some embodiments, the tripeptide is tripeptide-1. In some embodiments, a first hexapeptide of the one or more hexapeptides is hexapeptide-12. In some embodiments, a second hexapeptide of the one or more hexapeptides is hexapeptide-38.

The peptide can be functionalized. For example, the peptide can be functionalized with a fatty acid, e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. Examples include palmitoyl hexapeptide-12 (Pal-VGVAPG), palmitoyl tripeptide-1 (Pal-GHK), myristoyl hexapeptide-12 (Myr-VGVAPG), myristoyl tripeptide-1 (Myr-GHK). Palmitoyl or myristoyl functionalization can be desirable in certain embodiments as it exhibits enhanced penetration when compared to other fatty acids. In some embodiments, the peptide is functionalized with a chemical group. For example, the peptide is functionalized with acetyl. Examples include acetyl hexapeptide-38. In some instances, the peptide is functionalized with a functional group comprising no more than 14 carbons. In some instances, the peptide is functionalized with a functional group comprising no more than 3, 4, 5, 6, 7, 8, 9, 10, 11, 12, 13, 14, 15, 16, 17, 18, 19, 20, or more than 20 carbons. In some instances, the peptide is non-palmitoylated. Without wishing to be limited to a particular theory, incorporation of the peptide in a liposome, in some embodiments, increases the lipophilicity of a peptide that is functionalized or is not functionalized.

Some embodiments of the methods and compositions provided herein include as a first peptide glycine-histidine-lysine (GHK). GHK is a peptide sequence that is rarely found in the class of proteins in general, but is frequently found in extracellular matrix proteins. The small size of GHK permits it to approach membrane receptors far more easily than larger peptides. Further, its unique, copper-binding structure enhances copper transport into and out of cells and promotes wound healing through several different but related pathways. Due to its strong copper binding structure, GHK can be provided in the form of GHK-Cu (copper-bound GHK form).

In compositions, the tripeptide is typically present in an amount of from about 50 ppm or less to about 100, 200, 300, 400, or 500 ppm or more, e.g., 50 ppm to 150 ppm.

In compositions, the hexapeptide is typically present in an amount of from about 50 ppm or less to about 100, 200, 300, 400, or 500 ppm or more, e.g., 50 ppm to 150 ppm.

The peptides can advantageously be provided in a base for suitable for combining with other components of a liposomal composition. The base can include one or more components such as a thickener/binding agent (e.g., pentaerythrityl tetraisostearate), an emollient/dispersing agent (e.g., caprylic/capric triglyceride), a solvent (e.g., propylene carbonate), and/or a rheology modifier/antisettling agent (e.g., disteardimonium hectorite).

Metabolic Influencers of dWAT Lipolysis

Various metabolites and metabolic pathways may influence dWAT size. In some embodiments, lipolysis and fatty acid release may increase dWAT size. In some embodiments, amino acids may increase dWAT size and thereby stimulate hair growth. In some embodiments, the compositions disclosed herein comprise metabolic modulators of dWAT lipolysis. In some embodiments, the metabolic modulator of dWAT lipolysis is an amino acid. In one aspect, the amino acid is 1-carnitine. In some embodiments, L-carnitine may stimulate hair growth by increasing energy supply to the energy-consuming anagen hair matrix. In some embodiments, L-carnitine may stimulate human scalp hair growth by upregulation of proliferation and down regulation of apoptosis in follicular keratinocytes in vitro. In some embodiments, L-carnitine may work as a surrogate to lipolysis and amino acid release that is reduced when dWAT volume decreases.

Melatonin

Usually, melatonin, an antioxidant, may be associated with stimulation of hair growth. In some cases, melanotonin has positive effects resulting from the topical application of a melatonin solution in women and men with early-stage androgenic alopecia or general hair loss. In some embodiments, the compositions as disclosed herein comprise melatonin. In some embodiments, the compositions as disclosed herein comprise modulators of melatonin receptors. In some embodiments, the melatonin receptor is MT1, MT2, or RORα.

In some embodiments, stimulation of melatonin receptors may upregulate hair growth. Examples of such melatonin receptors may include but are not limited to MT1, MT2, and RORα.

Autophagy

Often, autophagy may be elevated as the HF progresses naturally through anagen, and may be instrumental to hair growth stimulation. Usually, autophagy induction can occur through various pathways. For example, the longevity metabolites α-ketobutyrate (α-KB) and α-ketoglutarate (α-KG), and activators of AMP-activated protein kinase (AMPK) can stimulate autophagy. In some caes, inhibitors of mammalian target of rapamycin (mTOR) can activate autophagy and thereby initiate hair follicle activation and hair regeneration.

Methods and compositions as disclosed herein may comprise components to stimulate autophagy in the hair follicle cell. In some embodiments, the component that stimulates autophagy is hexapeptide-11, α-KB, α-KG, 5-aminoimidazole-4-carboxamide ribonucleotide (AICAR), metformin, oligomycin, rapamycin, or combinations thereof. In some embodiments, the component that stimulates autophagy is an mTOR inhibitor. In some embodiments, the mTOR inhibitor is oligomycin. In some embodiments, the mTOR inhibitor is rapamycin. In some embodiments, the component that stimulates autophagy is an AMPK stimulator. In some embodiments, the AMPK stimulator is metformin.

Mitochondrial Pyruvate Carrier (MPC) Complex Inhibitors

Mitochondrial-derived energy source may influence hair follicle dormancy and mitochondrial pyruvate carrier (MPC) inhibition. Namely, MPC complex inhibitors could activate follicles that are dormant. In some embodiments, compositions as disclosed herein comprise an MPC complex inhibitor. In some embodiments, the MPC complex inhibitor is a member of the thiazolidinedione family. In one aspect, the member of the thiazolidinedione family is a glitazone.

Jak-STAT Inhibitors

Often, Jak-STAT signaling pathways are crucial for cellular signaling. Ususally, inhibition of Jak-STAT signaling may promote hair growth by stimulating the activation. In some caes, it may lead to proliferation of HF stem cells. In some cases, topical treatment with Jak-STAT inhibitors may result in more robust hair growth than systemic treatment in androgenic alopecia (AA). In some embodiments, compositions described herein comprise a Jak-STAT inhibitor. In some embodiments, the compositions described herein reduce the activity of Jak-STAT signaling pathway. In some embodiments, the compositions described herein inhibit the activity of Jak-STAT signaling pathway. In one aspect, the Jak-STAT inhibitor is a ruxolitinib. In one aspect, the Jak-STAT inhibitor is a tofacitinib.

Wnt/b-Catenin and BMP Signaling

Usually, the stimulatory Wnt/β-catenin signaling pathway and the inhibitory bone morphogenetic protein (BMP) signals may be involved in intrinsic activation of hair follicle stem cells (HF-SCs), or bulge stem cells, and the entry of hair follicles (HF) into anagen. Often, these signals arise from the DP that uphold HF-SCs in a quiescent state and generate synchronized cyclic waves of BMP activity that decline when Wnt expression waves arise, thereby controlling HF cycling. In some cases, HF growth stimulatory signals can be propagated during the transition from telogen to anagen via neighboring HFs. In some cases, Wnt signaling genes within the fibroblast growth factor family may be under-expressed and BMP signaling may be increased in aging dWAT. In some cases, several plant-derived chemicals may promote hair growth by activating Wnt/b-catenin signaling. In some embodiments, compositions as described herein comprise plant-derived chemicals that promote hair growth by activating Wnt/b-catenin signaling. In some aspects, the plant-derived chemicals include, but are not limited to, Prunus mira Koehne nut oil, red ginseng oil, and Loliolide.

Corticosterone and Stress

Provided herein are compositions and methods that reduce stress-induced inhibition of hair follicle stem cell (HF-SC) activation and hair growth. In some embodiments, the composition restores growth arrest-specific 6 (Gas6) expression. In some cases, stress may impact hair follicle status. In some cases, stress hormone corticosterone may regulate hair follicle stem cell (HF-SC) quiescence and hair growth. Under chronic stress, increased levels of corticosterone may prolong HF-SC quiescence and may maintain hair follicles in an extended resting phase. In some cases, corticosterone may act on the dermal papillae, suppressing the expression of a gene/protein called growth arrest-specific 6 (Gas6), which in turn has HF-SC receptors that stimulate hair growth. In some embodiments, restoring Gas6 expression overcomes the stress-induced inhibition of HF-SC activation and hair growth.

Fibroblast Growth Factor 5 (FGF5)

The compositions and methods provided herein may modulate the release of FGF5 by the perifollicular macrophages. In some cases, the modulation of release of FGF5 may increase entry of hair follicle into anagen. In some cases, the modulation is an increase in release of FGF5. Usually, mature HFs may have a distinct immune system. Often, the HF bulb and the HF bulge may represent areas of immune privilege, whose collapse gives rise to distinct inflammatory hair loss disorders. For example, perifollicular mast cells and macrophages have been implicated in the regulation of HF growth through anagen and the entry into catagen. In some cases, timed release of the fibroblast growth factor 5 (FGF5) by perifollicular macrophages may regulate the anagen-catagen switch. In some cases, changes in the release of Wnt signals by perifollicular macrophages may contribute to the establishment of the refractory and competent phases of telogen, and to the propagation of cues that induce anagen.

Penetration Enhancers

Various types of penetration enhancers may be used with the compositions as described herein. In some embodiments, the penetration enhancer comprises fatty acids, terpenes, alcohols, pyrrolidone, sulfoxides, laurocapram, surface active agents, amides, amines, lecithin, polyols, quaternary ammonium compounds, silicones, alkanoates, or combinations thereof.

Fatty acids and alcohols can be employed to enhance penetration of the peptides, and to provide a silky feel to formulations, e.g., methanoic acid, ethanoic acid, propanoic acid, butanoic acid, isobutyric acid, pentanoic acid, hexanoic acid, heptanoic acid, octanoic acid, nonanoic acid, decanoic acid, myristoleic acid, isovaleric acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, medium chain fatty acids, e.g., C₆₋₁₂ fatty acids, or the like. Typical amounts when employed in compositions are from 1% by weight to 4% by weight.

Other Components

Other components can include anti-inflammatory agents, antioxidants, plant-derived chemicals, and solubility enhancers. Exemplary anti-irritation agents include, but are not limited to, panthenyl triacetate and naringenin. Panthenyl triacetate and naringenin are natural plant extracts that reduce redness and water loss through the skin. Typical amounts for anti-irritation agents when employed in compositions are from 1% by weight to 4% by weight.

Exemplary anti-inflammatory agents include, but are not limited to, Arnica montana extract. Arnica montana extract includes components such as essential oils, fatty acids, thymol, pseudoguaianolide sesquiterpene lactones and flavanone glycosides. It can exhibit an anti-inflammatory effect. Typical amounts for anti-inflammatory agents when employed in compositions are from 1% by weight to 4% by weight.

Exemplary antioxidant agents include, but are not limited to, niacinamide. Niacinamide decreases the protein expression level of DKK-1 which appears to be hydrogen peroxide-induced by decreasing intracellular reactive oxidative species (ROS) production in dermal papilla cells. Thus, niacinamide could enhance hair growth by preventing oxidative stress-induced cell senescence and premature catagen entry of hair follicles. In some embodiments, niacinamide is provided at least or about 0.001%, 0.005%, 0.01%, 0.02%, 0.05%, 0.20%, 0.25%, 0.50%, 0.75%, 1.0%, 1.5%, 2.0%, 2.5%, 3.0%, 3.5%, 4.0%, or more than 4%. In some embodiments, Dunaliella salina extract is provided at about 2%.

Exemplary antioxidant agents include, but are not limited to, Dunaliella salina extract. Dunaliella salina extract includes components such as beta carotenes. It can exhibit an antioxidant effect. Typical amounts for anti-inflammatory agents when employed in compositions are from 0.1% by weight to 2% by weight. In some embodiments, Dunaliella salina extract is provided at least or about 0.001%, 0.005%, 0.01%, 0.02%, 0.05%, 0.10%, 0.25%, 0.50%, 0.75%, 1.0%, 1.5%, 2.0%, 2.5%, 3.0%, 3.5%, 4.0%, or more than 4%. In some embodiments, Dunaliella salina extract is provided at about 0.0027%.

Exemplary antioxidant agents include, but are not limited to, piroctone olamine, zinc pyrithione, zinc carbonate, niacinamide, panthenol and caffeine.

Certain components of the formulation tend to be difficult to solubilize in conventional formulations. Phosphatidylserine and oleuropein are known to exhibit solubility issues. In some embodiments, a siloxane polymer, e.g., caprylyl methicone, is used to solubilize phosphatidylserine. In some embodiments, caprylyl methicone is used to solubilize phosphatidylserine in anhydrous formulations. In some embodiments, panthenyl triacetate and naringenin is used to solubilize oleuropein. For topical compositions containing from about by weight to about 0.1% by weight phosphatidylserine and/or from about 0.05% by weight to about 0.1% by weight oleuropein, caprylyl methicone in an amount of from about 0.5% by weight to about 1% by weight of caprylyl methicone can solubilize phosphatidylserine in an anhydrous formulation. In some embodiments, phosphatidylserine is provided at least or about 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4%. In some embodiments, the phosphatidylserine is provided at about 0.05% by weight. In some embodiments, the phosphatidylserine is provided at about 0.25% by weight. In some embodiments, the phosphatidylserine is provided at about 1% by weight. In some embodiments, the oleuropein is provided at least or about 0.001%, 0.005%, 0.01%, 0.02%, 0.05%, 0.10%, 0.20%, 0.25%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the oleuropein is provided in a range of about 0.001% to about 6%, about 0.002% to about 4%, about 0.01% to about 3%, about 0.02% to about 2%, or about 0.01% to about 0.05% by weight. In some embodiments, the oleuropein is provided at about 0.010% by weight. In some embodiments, the oleuropein is provided at about 0.020% by weight. In some embodiments, the oleuropein is provided at about 0.050% by weight.

Bentonite clays can be employed in conjunction with the peptides to provide impart penetration and adsorption properties to the compositions, and can aid in stabilizing emulsions. Other clays, such as hectorite and magnesium aluminum silicate can also be employed. Bentonite or other clays can be modified to yield an organic modified clay compound. Salts (e.g., quaternary ammonium salts) of fatty acids (e.g., hydrogenated fatty acids) can be reacted with hectorite or other clays. As provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δn, where n indicates the lower numbered carbon of each pair of double-bonded carbon atoms. A shorthand notation specifying total #carbons:#double bonds, A double bond positions can be employed. For example, 20:4Δ_{5,8,11,14} refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-49-octadecenoate) is a monounsaturated fatty acid, linolenate (all-cis-49,12,15-octadecatrienoate) is a polyunsaturated fatty acid. Fatty acids suitable for use can comprise from 5 to 30 carbon atoms, e.g., 5, 6, 7, 8, 9, 10, 11, 12, 13, 14, 15, 16, 17, 18, 19, 20, 21, 22, 23, 24, 25, 26, 27, 28, 29, or 30 carbon atoms. The fatty acid can be fully saturated, or can include as many double bonds as are feasible for the chain length. Fatty acids suitable for functionalizing hectorite or other clays include palmitic acid and stearic acid. Dialkyl quaternary cationic modifiers include dipalmoyldimonium chloride and distearyldimonium chloride. Amidoamine quaternary cationic modifiers include palmitamidopropyltrimonium chloride cetearyl alcohol and palmitamidopropyltrimonium chloride.

In some embodiments, the peptides can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for compositions include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the peptide compositions as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the peptides into the skin. An alternative to increasing molecular weight (as with silicone gums) or adding filler (as with silicone compounds) is to partially crosslink siloxane polymers and disperse this material in an appropriate silicone carrier fluid. The resulting dimethicone crosspolymers (also known as silicone elastomers in the personal care industry) differ from basic polydimethylsiloxane (PDMS) because of the cross-linking between the linear polymers. These materials can be employed in peptide formulations, and also offer benefits in scar treatment, periwound protection and enzyme delivery. In skin care applications, the aesthetics of silicone elastomers (including those with functional groups) and their ability to absorb various oils (e.g., with a dimethicone/vinyl dimethicone crosspolymer such as Dow Corning® 9506 Elastomer Powder) are two of the elastomer's desirable properties. Silicone elastomers have a skin feel different from any of the silicone fluids, described as “smooth,” “velvety,” and “powdery.” It can be modified by controlling the amount of liquid phase in the formula, and therefore the degree of swelling. Due to their film-forming properties, dimethicone crosspolymers can be used as delivery systems for active ingredients such as the peptides described herein, or other formulation components such as oil-soluble vitamins and sunscreens. Sunscreens such as octyl methoxycinnamate can be more efficiently delivered from a formulation containing a silicone elastomer, producing a higher sun protection factor (SPF). Silicone elastomer blends can be used to enhance SPF in oil-in-water formulations containing organic sunscreens. For example, in testing conducted regarding SPF, the addition of 4% silicone elastomer blend to a suncare formulation containing organic sunscreens increased the SPF from 5.7 to 18. This property of the silicone elastomer allows the effectiveness of sunscreen agents in a formulation to be maximized while reducing the amount needed to achieve a desired SPF. As a result, formulation costs can be reduced along with potential irritation caused by sunscreen actives. Accordingly, a higher SPF can be achieved with the same amount of UV absorber, resulting in enhanced performance with no added formulation cost. Silicone elastomers can be produced from linear silicone polymers by a variety of crosslinking reactions, e.g., by a hydrosilylation reaction in which a vinyl group reacts with a silicon hydride. The general process involves linear silicone polymers with reactive sites along the polymer chain reacting with a cross-linker. The dimethicone crosspolymer can be produced either as a gel made of a suspension of elastomer particles swollen in a carrier fluid (e.g., a mixture of high molecular weight silicone elastomer in cyclopentasiloxane such as Dow Corning® 9040 Silicone Elastomer Blend), or as a spray-dried powder (a dimethicone/vinyl dimethicone crosspolymer such as Dow Corning® 9506 Elastomer Powder). The gel form having desirable attributes is cyclomethicone, but low viscosity dimethicones and organic fluids can also be used. Examples of dimethicone crosspolymers in the suspension or gel form are high molecular weight silicone elastomer (12%) in decamethylcyclopentasiloxane (e.g., Dow Corning® ST-Elastomer 10) and a mixture of high molecular weight silicone elastomer in cyclopentasiloxane (e.g., Dow Corning® 9040 Silicone Elastomer Blend), which typically have an elastomer content ranging from 10 to 20% by weight.

The pharmaceutical excipients used in the topical preparations of the peptide compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof

Suitable solvents for an aqueous or hydrophilic liposomal composition include water; ethyl alcohol; isopropyl alcohol; mixtures of water and ethyl and/or isopropyl alcohols; glycerin; ethylene, propylene or butylene glycols; DMSO; and mixtures thereof. In some embodiments, glycerin is provided at least or about 1%, 2%, 3%, 4%, 5%, 6%, 7%, 8%, 9%, 10%, 11%, 12%, or more than 12%. In some embodiments, glycerin is provided at least or about 7%. In some embodiments, glycerin is provided in a range of about 1% to about 12%, about 2% to about 11%, or about 3% to about 10%. Suitable solvents for hydrophobic compositions include mineral oils, vegetable oils, and silicone oils. If desired, the peptide compositions as described herein may be dissolved or dispersed in a hydrophobic oil phase, and the oil phase may then be emulsified in an aqueous phase comprising water, alone or in combination with lower alcohols, glycerin, and/or glycols. It is generally preferred to employ anhydrous compositions, as the presence of water can result in stinging upon administration to skin tissues subject to laser treatment, chemical peel, dermabrasion, or the like. Anhydrous formulations may also act to prevent the development of water-based irritant contact dermatitis in damaged or sensitive skin, which may produce rashes and skin irritation that may retard wound healing and improvement in skin quality. Tsai, T. F., Maibach, H. I. How irritant is water? An overview. Contact Dermatitis 41(6) (1999): 311-314 (describing contact dermatitis caused by water as an irritant). However, in certain embodiments it may be acceptable to provide water based compositions, or to permit a limited amount of water to be present. For example, water may be present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the compositions include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the compositions. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in compositions include anionic, cationic, nonionic, and zwitterionic surfactants. Preferred ionic emulsifiers include phospholipids, such as lecithin and derivatives.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in compositions include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in compositions include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The peptide compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

### Therapeutic Uses

Methods and compositions as described herein for targeting dermal white adipose tissue (dWAT) may result in hair follicle stimulation or regeneration. In some instances, methods and compositions as described herein promote hair growth. In some instances, methods and compositions as described herein are used for reducing or preventing alopecia.

Alopecia can comprise various types of hair loss. In some instances, the hair loss is male or female pattern baldness, alopecia areata, telogen effluvium, anagen effluvium, or combinations thereof. In some instances, hair loss is caused by an infection. Exemplary infections causing hair loss include, but are not limited to, dissecting cellulitis, fungal infections (e.g., tinea capitis), folliculitis, secondary syphilis, and demodex folliculorum. In some instances, hair loss is caused by drugs or medications (e.g., chemotherapy). In some instances, hair loss is caused by trauma including, but not limited to, traction alopecia, frictional alopecia, trichotillomania, radiation, chemotherapy, and surgery. In some instances, hair loss is caused by a disease or disorder. In some instances, hair loss is caused by alopecia mucinosa, biotinidase deficiency, chronic inflammation, diabetes, lupus erythematosus, pseudopelade of Brocq, telogen effluvium, tufted folliculitis, and genetics.

In some embodiments, the methods and compositions described herein are used in conjunction with a hair loss treatment. In some embodiments, the hair loss treatment is non-invasive. In some instances, the hair loss treatment is invasive. In some instances, the hair loss treatment comprises topical administration of a drug. In some instances, the hair loss treatment comprises ingestion of a drug. In some instances, the hair loss treatment comprises a hair replacement procedure, scalp reduction, or combinations thereof. Exemplary hair replacement procedures include, but are not limited to, micro-grafting, slit grafting, and punch grafting. In some instances, the hair loss treatment comprises use of platelet-rich plasma.

In some instances, the compositions described herein are administered once per day, twice per day, three times per day or more. The compositions described herein, in some embodiments, are administered daily, every day, every alternate day, five days a week, once a week, every other week, two weeks per month, three weeks per month, once a month, twice a month, three times per month, or more. In some embodiments, the compositions described herein are administered twice daily administration, e.g., morning and evening. In some embodiments, the liposomal compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more.

In some instances, the compositions as described herein are administered to a site of hair loss. In some instances, the compositions as described herein are administered to a site to prevent hair loss. Hair loss can occur on any part of the body. In some instances, the compositions as described herein are administered to a site to promote hair growth.

Compositions as described herein may be administered prior to a hair loss treatment. In some instances, the compositions described herein are administered up to 1 day, up to 2 days, up to 3 days, up to 5 days, or more than 5 days prior to a hair loss treatment. Sometimes the compositions described herein are administered singly, or over a time course, such as daily, multiple times weekly, weekly, biweekly, monthly or less frequently prior to a hair loss treatment. In some instances, the compositions described herein are administered singly, or over a time course, such as daily, multiple times weekly, weekly, biweekly, monthly or more frequently prior to a hair loss treatment.

Compositions as described herein may be administered during a hair loss treatment.

Compositions as described herein may be administered following a hair loss treatment. In some instances, the compositions described herein are administered up to 1 day, up to 2 days, up to 3 days, up to 5 days, or more than 5 days following a hair loss treatment. Sometimes the compositions described herein are administered singly, or over a time course, such as daily, multiple times weekly, weekly, biweekly, monthly or less frequently following a hair loss treatment. In some instances, the compositions described herein are administered singly, or over a time course, such as daily, multiple times weekly, weekly, biweekly, monthly or more frequently following a hair loss treatment.

### Stability Testing

Stability testing of the compositions can be conducted as follows.

High temperature testing is now commonly used as a predictor of long-term stability. High temperature testing can be conducted at 37° C. (98 F) and 45° C. (113° F.). If a product is stored at 45° C. for three months (and exhibits acceptable stability) then it should be stable at room temperature for two years. A good control temperature is 4° C. (39° F.) where most products will exhibit excellent stability. Sometime, the product is also be subjected to −10° C. (14° F.) for three months.

In some instances, stability of the product is assessed by passing three cycles of temperature testing from −10° C. (14° F.) to 25° C. (77° F.). In such cases, the product is placed at −10° C. for 24 hours and then placed at room temperature (25° C.) for 24 hours. This completes one cycle. An even more rigorous test is a −10° C. to 45° C. five-cycle test. This puts emulsions under a tremendous stress.

The dispersed phase (of an oil-in-water emulsion) has a tendency to separate and rise to the top of the emulsion forming a layer of oil droplets. This phenomenon is called creaming. Creaming is one of the first signs of impending emulsion instability. A test method to predict creaming is centrifugation. Heat the emulsion to 50° C. (122° F.) and centrifuge it for thirty minutes at 3000 rpm. Then inspect the resultant product for signs of creaming.

Both formulas and packaging can be sensitive to the UV radiation. The product is placed in glass and the actual package in a light box that has a broad-spectrum output. Another glass jar completely covered with aluminum foil serves as a control. Discoloration of the product may be observed.

For all the above mentioned tests the color, odor/fragrance, viscosity, pH value, and, if available, particle size uniformity and/or particle agglomeration under the microscope can be observed.

### Embodiments

Numbered embodiment 1 comprises a composition for targeting dermal white adipose tissue (dWAT) for stimulating or regenerating a hair follicle comprising: (a) a tripeptide-1; (b) a hexepaptide-12; and (c) a PPAR-gamma agonist, wherein the composition stimulates or regenerates the hair follicle. Numbered embodiment 2 comprises the composition of numbered embodiment 1, wherein the tripeptide-1 comprises palmitoyl tripeptide-1, myristoyl tripeptide-1, or a combination thereof. Numbered embodiment 3 comprises the composition of numbered embodiment 1, wherein the hexapeptide-12 comprises palmitoyl hexapeptide-12, myristoyl hexapeptide-12, or a combination thereof. Numbered embodiment 4 comprises the composition of numbered embodiment 1, further comprising hexapeptide-38. Numbered embodiment 5 comprises the composition of numbered embodiment 4, wherein the hexapeptide-38 is acetyl-hexapeptide-38. Numbered embodiment 6 comprises the composition of numbered embodiment wherein the hexapeptide-38 is encapsulated in a liposome. Numbered embodiment 7 comprises the composition of numbered embodiment 1, further comprising an octapeptide. Numbered embodiment 8 comprises the composition of numbered embodiment 1, wherein the PPAR-gamma agonist is thiazolidinedione (TZD), aleglitazar, farglitazar, muraglitazar, tesaglitazar, or combinations thereof. Numbered embodiment 9 comprises the composition of numbered embodiment 1, wherein the PPAR-gamma agonist is derived from a plant source. Numbered embodiment 10 comprises the composition of numbered embodiment 1, wherein the PPAR-gamma agonist is a natural product. Numbered embodiment 11 comprises the composition of numbered embodiment 1, further comprising a M2-macrophage-polarizing compound. Numbered embodiment 12 comprises the composition of numbered embodiment 11, wherein the M2-macrophage-polarizing compound is a flavonoid, terpenoid, glycoside, lignan, coumarin, alkaloid, polyphenol, quinone, or combinations thereof. Numbered embodiment 13 comprises the composition of numbered embodiment 11, wherein the M2-macrophage-polarizing compound is Arctigenin. Numbered embodiment 14 comprises the composition of numbered embodiment 11, wherein the M2-macrophage-polarizing compound is lupeol, malibatol A, geraniin, aloe-emodin, quercetin, curcumin, apigenin, tacrolimus, or niacinamide, or combinations thereof. Numbered embodiment 15 comprises the composition of numbered embodiment 11, wherein the M2-macrophage-polarizing compound is derived from a plant source. Numbered embodiment 16 comprises the composition of numbered embodiment 15, wherein the plant source is Arctium lappa plant, curcumin, luteolin, piperine, resveratrol, silibinum, Baicalin or combinations thereof. Numbered embodiment 17 comprises the composition of numbered embodiment 1, further comprising a topical activator of insulin growth factor 1 (IGF-1). Numbered embodiment 18 comprise the composition of numbered embodiment 17, wherein the topical activator of insulin growth factor 1 (IGF-1) is capsaicin, isoflavone, or combinations thereof. Numbered embodiment 19 comprises the composition of numbered embodiment 17, wherein the topical activator of insulin growth factor 1 (IGF-1) is capsaicin. Numbered embodiment 20 comprise the composition of numbered embodiment 1, further comprising an activator of PGC-la. Numbered embodiment 21 comprises the composition of claim 1, wherein the PPAR-gamma agonist comprises adiponectin or an adiponectin mimetic. Numbered embodiment 22 comprises the composition of claim 21, wherein the adiponectin mimetic comprises AdipoRon, ADP355, ADP399, JT003, 6-C-O-D-glucopyranosyl-(2S,3S)-(+)-5, 7, 30, 40-tetrahydroxydihydroflavonol (GTDF), Osmotin, or combinations thereof. Numbered embodiment 23 comprises the composition of claim 1, further comprising a Jak-STAT inhibitor. Numbered embodiment 24 comprises the composition of claim 23, wherein the Jak-STAT inhibitor comprises a ruxolitinib, tofacitinib, or combinations thereof. Numbered embodiment 25 comprises a method for stimulating or regenerating a hair follicle comprising administering the composition of numbered embodiment 1. Numbered embodiment 26 comprises the method of numbered embodiment 25, wherein the method stimulates hair growth. Numbered embodiment 27 comprises a method for preventing or reducing hair loss comprising administering the composition of numbered embodiment 1.

What we claim is:

1. A composition for targeting dermal white adipose tissue (dWAT), the composition comprising:
a tripeptide-1;
a hexepaptide-12; and
a PPAR-gamma agonist.

2. The composition of claim 1, wherein the tripeptide-1 comprises palmitoyl tripeptide-1, myristoyl tripeptide-1, or a combination thereof.

3. The composition of claim 1, wherein the hexapeptide-12 comprises palmitoyl hexapeptide-12, myristoyl hexapeptide-12, or a combination thereof.

4. The composition of claim 1, further comprising one of a hexapeptide-38 or a hexapeptide-11.

5. The composition of claim 1, further comprising an octapeptide.

6. The composition of claim 5, wherein the octapeptide is GPHGVRQA.

7. The composition of claim 1, wherein the PPAR-gamma agonist comprises thiazolidinedione (TZD), aleglitazar, farglitazar, muraglitazar, tesaglitazar, pioglitazone, rosiglitazone, rivoglitazone, troglitazone, or a combination thereof.

8. The composition of claim 1, wherein the PPAR-gamma agonist comprises adiponectin or an adiponectin mimetic.

9. The composition of claim 8, wherein the adiponectin mimetic comprises one or more selected from the group consisting of: AdipoRon; ADP355; ADP399; JT003; 6-C-O-D-glucopyranosyl-(2S,3S)-(+)-5,7,30,40-tetrahydroxydihydroflavonol (GTDF); and osmotin.

10. The composition of claim 1, further comprising a M2-macrophage-polarizing compound.

11. The composition of claim 10, wherein the M2-macrophage-polarizing compound comprises a flavonoid, terpenoid, glycoside, lignan, coumarin, alkaloid, polyphenol, quinone, or combinations thereof.

12. The composition of claim 10, wherein the M2-macrophage-polarizing compound comprises Arctigenin lupeol, malibatol A, geraniin, aloe-emodin, quercetin, curcumin, apigenin, tacrolimus, niacinamide, luteolin, piperine, resveratrol, silibinum, Baicalin, or a combination thereof.

13. The composition of claim 1, further comprising a topical activator of insulin growth factor 1 (IGF-1).

14. The composition of claim 13, wherein the topical activator of insulin growth factor 1 (IGF-1) comprises capsaicin, isoflavone, or combinations thereof.

15. The composition of claim 1, further comprising an activator of PGC-la.

16. The composition of claim 1, further comprising a Jak-STAT inhibitor.

17. The composition of claim 16, wherein the Jak-STAT inhibitor comprises a ruxolitinib, tofacitinib, or combinations thereof.

18. The composition of claim 1, wherein the composition further comprises L-carnitine, melatonin, Prunus mira Koehne nut oil, red ginseng oil, loliolide, hexapeptide-11, α-ketobutyrate (α-KB), α-ketoglutarate (α-KG), 5-aminoimidazole-4-carboxamide ribonucleotide (AICAR), metformin, oligomycin, rapamycin, Vitamin D, capsaicin, isoflavone, vascular endothelial growth factor (VEGF), TGF-β, or a combination thereof.

19. The composition of claim 1, wherein the composition stimulates or regenerates a hair follicle.

20. A method for stimulating or regenerating a hair follicle, the method comprising topically administering a composition comprising:
a tripeptide-1;
a hexepaptide-12; and
a PPAR-gamma agonist.

21. A method for preventing or reducing hair loss, the method comprising topically administering a composition comprising:
a tripeptide-1;
a hexepaptide-12; and
a PPAR-gamma agonist.
